(19) United States
(12) Patent Application Publication
(10) Pub. No.: US 2008/0234810 A1
(43) Pub. Date: Sep. 25, 2008

(54) Amorphous Glass-Coated Drug Delivery Medical Device

(75) Inventors: James M. Carlson, Bloomington, IN (US); Ni Ding, San Jose, CA (US)

Correspondence Address: SQUIRE, SANDERS & DEMPSEY LLP, 1 MARITIME PLAZA, SUITE 300, SAN FRANCISCO, CA, 94111, US

(73) Assignee: Abbott Cardiovascular Systems Inc., Santa Clara, CA (US)

(21) Appl. No.: 11/769,581

(22) Filed: Jun. 27, 2007

Related U.S. Application Data
(60) Provisional application No. 60/817,485, filed on Jun. 28, 2006.

(51) Int. Cl. A61F 2/82; C23C 16/453

(52) U.S. Cl. 623/1.42; 623/1.46; 427/2.25; 977/780

(57) ABSTRACT

An implantable medical device that can include an amorphous glass primer layer, an amorphous glass drug-containing layer and a nanoporous amorphous glass top-coat layer is disclosed.

### RELATED APPLICATION

This application claims benefit of and incorporates by reference U.S. Provisional Patent Application No. 60/817,485 which was filed on Jun. 28, 2006.

### FIELD OF THE INVENTION

The present invention is directed to an amorphous glass-coated drug delivery medical device.

### BACKGROUND OF THE INVENTION

New compositions and methods to improve and control the properties of medical devices are continually being sought. This is particularly true for medical devices that can be implanted in a patient, e.g., a stent, where predictable and controllable performance is essential to successful treatment.

Stents act as a mechanical means to physically hold open and, if desired, expand a passageway in a patient. In practice, a stent is typically compressed, inserted into a small vessel through a catheter, and then expanded to a larger diameter once placed in a proper location. Stents play an important role in a variety of medical procedures such as, for example, percutaneous transluminal coronary angioplasty (PTCA). Stents are generally implanted in such procedures to reduce occlusion formation and restenosis and to maintain patency within vascular lumens.

Restenosis or reclosure of the artery has been an ongoing challenge with the use of bare metal stents. In order to decrease restenosis, stent manufacturers have been experimenting with applying anti-restenotic drugs onto the stents. The common drug coating designs consist of a primer layer, followed by a drug layer or drug/polymer layer and an optional topcoat layer containing pure polymer or a mixture of drug/polymer. However, current methods of dispersing an active agent in a polymer or attaching an active agent to a polymer often result in a drug coating morphology that sometimes is difficult to predict and control. This makes delivery of the agent less predictable.

In certain situations, manufacturing inconsistencies among different stents can arise which have the potential for release-rate variability or compromise coating integrity. For example, when a polymeric matrix is used as a primer layer, inadequate adhesion between a drug coat and the polymeric matrix primer layer is sometimes observed. Indeed, depending upon the nature of the primer, a primer layer can delaminate and/or flake off in an unpredictable manner, which among other issues, affects the coating integrity and the ability to tightly control drug release rate. Similarly, polymeric drug reservoir layers and topcoat layers can also increase release rate variability.

Accordingly, there is a need for suitable non-polymeric primer layers, drug reservoir layers and topcoat layers for use in the production of drug-coated medical devices, thereby allowing for suitable and precise control over the release rates of agents and their subsequent uptake by local tissues.

### SUMMARY OF THE INVENTION

The present invention relates to an implantable medical device that includes a device body, an optional amorphous glass primer layer, a reservoir layer that includes one or more bioactive agents disposed over the device body and the amorphous glass primer layer if selected and an optional nanoporous amorphous glass top-coat layer disposed over the reservoir layer, wherein if the amorphous glass primer layer is not selected the nanoporous amorphous glass top-coat layer must be present and wherein if the nanoporous amorphous glass top-coat layer is not selected the amorphous glass primer layer must be present. In one aspect, the device body is a stent.

In various aspects, the stent material is selected from a group that includes stainless steel, nitinol, tantalum, tantalum alloy, titanium, titanium alloy, cobalt chromium, alloy x, niobium, niobium alloy, zirconium and zirconium alloy.

In various aspects, the bioactive agent is selected from a group that includes a corticosteroid, everolimus, an everolimus derivative, zotarolimus, a zotarolimus derivative, sirolimus, a sirolimus derivative, paclitaxel, a bisphosphonate, ApoA1, a mutated ApoA1, ApoA1 milano, an ApoA1 mimetic peptide, an ABC A1 agonist, an anti-inflammatory agent, an anti-proliferative agent, an anti-angiogenic agent, a matrix metalloproteinase inhibitor and a tissue inhibitor of metalloproteinase.

In various aspects, the reservoir layer is composed of a polymeric matrix or amorphous glass.

In various aspects, the diameter of the nanopores in the nanoporous amorphous glass top-coat layer is no larger than 100 nanometers, 75 nanometers, 50 nanometers, 25 nanometers or 10 nanometers.

Another aspect of the present invention relates to a method of coating an implantable medical device. The method involves providing an implantable medical device, applying an amorphous glass primer layer to the implantable medical device, applying a reservoir layer material that includes one or more bioactive agents over the amorphous glass primer layer and the implantable medical device, applying a nanoporous amorphous glass top-coat layer over the reservoir layer material and forming a coating comprising the amorphous glass primer layer, the reservoir layer and the nanoporous amorphous glass top-coat layer on the implantable medical device.

In various aspects, applying the amorphous glass primer and the nanoporous amorphous glass top-coat layer involves chemical vapor deposition.

In one aspect, the implantable medical device is a stent.

In various aspects, the stent material is selected from a group that includes stainless steel, nitinol, tantalum, tantalum alloy, titanium, titanium alloy, cobalt chromium, alloy x, niobium, niobium alloy, zirconium and zirconium alloy.

In various aspects, the bioactive agent is selected from a group that includes a corticosteroid, everolimus, an everolimus derivative, zotarolimus, a zotarolimus derivative, sirolimus, a sirolimus derivative, paclitaxel, a bisphosphonate, ApoA1, a mutated ApoA1, ApoA1 milano, an ApoA1 mimetic peptide, an ABC A1 agonist, an anti-inflammatory agent, an anti-proliferative agent, an anti-angiogenic agent, a matrix metalloproteinase inhibitor and a tissue inhibitor of metalloproteinase.

In various aspects, the reservoir layer material is composed of a polymeric matrix or amorphous glass.

In various aspects, the diameter of the nanopores in the nanoporous amorphous glass top-coat layer is no larger than 100 nanometers, 75 nanometers, 50 nanometers, 25 nanometers or 10 nanometers.

Another aspect of the present invention relates to an implantable medical device that includes a device body, wherein the device body includes a stent made of a material selected from the group that includes stainless steel, nitinol, tantalum, tantalum alloy, titanium, titanium alloy, cobalt chromium, alloy x, niobium, niobium alloy, zirconium and zirconium alloy. The medical device further includes an optional amorphous glass primer layer and a reservoir layer that includes a polymer and one or more bioactive agents disposed over the device body and the amorphous glass primer layer if selected, wherein the bioactive agent is selected from the group that includes a corticosteroid, everolimus, an everolimus derivative, zotarolimus, a zotarolimus derivative, sirolimus, a sirolimus derivative, paclitaxel, a bisphosphonate, ApoA1, a mutated ApoA1, ApoA1 milano, an ApoA1 mimetic peptide, an ABC A1 agonist, an anti-inflammatory agent, an anti-proliferative agent, an anti-angiogenic agent, a matrix metalloproteinase inhibitor and a tissue inhibitor of metalloproteinase. The medical device further includes an optional nanoporous amorphous glass top-coat layer, having nanopores with a diameter no larger than 100 nanometers disposed over the reservoir layer, wherein if the amorphous glass primer layer is not selected the nanoporous amorphous glass top-coat layer must be present and if the nanoporous amorphous glass top-coat layer is not selected the amorphous glass primer layer must be present.

Another aspect of the present invention relates to an implantable medical device that includes a device body, wherein the device body includes a stent made of a material selected from the group that includes stainless steel, nitinol, tantalum, tantalum alloy, titanium, titanium alloy, cobalt chromium, alloy x, niobium, niobium alloy, zirconium and zirconium alloy. The medical device further includes an optional amorphous glass primer layer and a reservoir layer that includes an amorphous glass and one or more bioactive agents disposed over the device body and the amorphous glass primer layer if selected, wherein the bioactive agent is selected from the group that includes a corticosteroid, everolimus, an everolimus derivative, zotarolimus, a zotarolimus derivative, sirolimus, a sirolimus derivative, paclitaxel, a bisphosphonate, ApoA1, a mutated ApoA1, ApoA1 milano, an ApoA1 mimetic peptide, an ABC A1 agonist, an anti-inflammatory agent, an anti-proliferative agent, an anti-angiogenic agent, a matrix metalloproteinase inhibitor and a tissue inhibitor of metalloproteinase. The device further includes an optional nanoporous amorphous glass top-coat layer, having nanopores with a diameter no larger than 100 nanometers disposed over the reservoir layer, wherein, if the amorphous glass primer layer is not selected the nanoporous amorphous glass top-coat layer must be present and if the nanoporous amorphous glass top-coat layer is not selected the amorphous glass primer layer must be present.

### BRIEF DESCRIPTION OF THE DRAWINGS

FIG. 1 depicts a device for coating an implantable medical device with an amorphous glass primer layer, reservoir layer or topcoat layer.

FIG. 2 is a micrograph showing an amorphous glass coating at 128,000× illustrating the presence of nanopores.

FIG. 3 shows energy dispersive x-ray analysis of an amorphous glass coated stent confirming the chemical composition of the coating to be silicon dioxide.

FIGS. 4A-C are scanning electron micrographs showing an amorphous glass-coated Vision™ stent that was balloon expanded to 4.5 mm diameter at 100×, 200×, and 400×, respectively.

### DETAILED DESCRIPTION

The present invention provides an implantable medical device that includes a device body, an optional amorphous glass primer layer, a reservoir layer that includes one or more bioactive agents disposed over the device body and the amorphous glass primer layer if selected and an optional nanoporous amorphous glass top-coat layer disposed over the reservoir layer if selected. In various aspects, the reservoir layer is composed of a polymeric matrix or amorphous glass.

As used herein, “implantable medical device” refers to any suitable medical substrate that can be implanted in a human or veterinary patient. Examples of such implantable devices include, but are not limited to, self-expandable stents and balloon-expandable stents.

The underlying structure of the device can be of virtually any design. The device can be made of a metallic material or an alloy such as, but not limited to, cobalt chromium alloy (ELGILOY), stainless steel (316L), high nitrogen stainless steel, e.g., BIODUR 108, cobalt chrome alloy L-605, “MP35N,” “MP20N,” ELASTINITE (Nitinol), tantalum, nickel-titanium alloy, platinum-iridium alloy, gold, magnesium or a combination thereof. “MP35N” and “MP20N” are trade names for alloys of cobalt, nickel, chromium and molybdenum available from Standard Press Steel Co., Jenkintown, Pa. “MP35N” consists of 35% cobalt, 35% nickel, 20% chromium, and 10% molybdenum. “MP20N” consists of 50% cobalt, 20% nickel, 20% chromium, and 10% molybdenum. The device can also be made of one or more polymers, including, but not limited to, poly-ester and poly-1-lactide.

Presently preferred device materials include stainless steel, nitinol, tantalum, tantalum alloy, titanium, titanium alloy, cobalt chromium, alloy x, niobium, niobium alloy, zirconium and zirconium alloy. Alloy x refers to a nickel-chromium-iron-molybdenum alloy.

As used herein, “primer layer” refers to a coating that exhibits good adhesion characteristics and good biocompatibility with regard to the material of which a medical device, i.e., a stent, is manufactured, and good adhesion characteristics with regard to whatever material is to be coated on the medical device, e.g., a drug-containing matrix. Preferably, an amorphous glass layer is employed as the primer layer for use with the devices and methods of the present invention.

As used herein, “amorphous glass” refers to silicon oxides exhibiting short range atomic order and no crystallinity.

As used herein, “reservoir layer” refers to a layer that acts as a reservoir or depot for a bioactive agent of the invention. The reservoir layer can be composed of a polymeric matrix, amorphous glass or can consist of bioactive agent only.

As used herein, “polymeric matrix” refers to a polymer structure that can act as a reservoir for a bioactive agent.

The polymeric matrix which is disposed over an amorphous glass primer layer of the invention is generally a biocompatible polymer that can be biostable or biodegradable and can be hydrophobic or hydrophilic. Suitable polymers are known to those skilled in the art.

As used herein, “biocompatible” refers to a polymer that both in its intact, as synthesized state and in its decomposed state, i.e., its degradation products, is not, or at least is minimally, toxic to living tissue; does not, or at least minimally and reparably, injure(s) living tissue; and/or does not, or at least minimally and/or controllably, cause(s) an immunological reaction in living tissue.

In various aspects, the reservoir layer will contain one or more bioactive agents which can be released from the medical device.

The bioactive agent, also referred to herein as a drug, can be an antiproliferative agent, an anti-inflammatory agent, an antineoplastic, an antimitotic, an antiplatelet, an anticoagulant, an antifibrin, an antithrombin, a cytostatic agent, an antibiotic, an anti-allergic agent, an anti-enzymatic agent, an angiogenic agent, a cyto-protective agent, a cardioprotective agent, a proliferative agent, an ABC A1 agonist or an antioxidant.

Suitable antiproliferative agents include, without limitation, actinomycin D, or derivatives or analogs thereof, i.e., actinomycin D is also known as dactinomycin, actinomycin IV, actinomycin I₁, actinomycin X₁, and actinomycin C₁. Antiproliferative agents can be natural proteineous agents such as a cytotoxin or a synthetic molecule, all taxoids such as taxols, docetaxel, and paclitaxel, paclitaxel derivatives, all olimus drugs such as macrolide antibiotics, rapamycin, everolimus, structural derivatives and functional analogues of rapamycin, structural derivatives and functional analogues of everolimus, FKBP-12 mediated mTOR inhibitors, biolimus, perfenidone, prodrugs thereof, co-drugs thereof, and combinations thereof. Representative rapamycin derivatives include 40-O-(3-hydroxy)propyl-rapamycin, 40-O-[2-(2-hydroxy)ethoxy]ethyl-rapamycin, or 40-O-tetrazole-rapamycin, 40-epi-(N-1-tetrazolyl)-rapamycin, prodrugs thereof, co-drugs thereof, and combinations thereof.

Suitable anti-inflammatory agents include, without limitation, steroidal anti-inflammatory agents, a nonsteroidal anti-inflammatory agent, or a combination thereof. In some embodiments, anti-inflammatory agents include clobetasol, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, prodrugs thereof, co-drugs thereof, and combinations thereof. The anti-inflammatory agent may also be a biological inhibitor of proinflammatory signaling molecules including antibodies to such biological inflammatory signaling molecules.

Suitable antineoplastics and/or antimitotics include, without limitation, paclitaxel, docetaxel, methotrexate, azathioprine, vincristine, vinblastine, fluorouracil, doxorubicin hydrochloride, and mitomycin.

Suitable antiplatelet, anticoagulant, antifibrin, and antithrombin drugs include, without limitation, sodium heparin, low molecular weight heparins, heparinoids, hirudin, argatroban, forskolin, vapiprost, prostacyclin, prostacyclin dextran, D-phe-pro-arg-chloromethylketone, dipyridamole, glycoprotein IIb/IIIa platelet membrane receptor antagonist antibody, recombinant hirudin and thrombin, thrombin inhibitors such as Angiomax ä (Biogen, Inc., Cambridge, Mass.), calcium channel blockers (such as nifedipine), colchicine, fish oil (omega 3-fatty acid), histamine antagonists, lovastatin (an inhibitor of HMG-CoA reductase, a cholesterol lowering drug, brand name Mevacor® from Merck & Co., Inc., Whitehouse Station, N.J.), monoclonal antibodies (such as those specific for Platelet-Derived Growth Factor (PDGF) receptors), nitroprusside, phosphodiesterase inhibitors, prostaglandin inhibitors, suramin, serotonin blockers, steroids, thioprotease inhibitors, triazolopyrimidine (a PDGF antagonist), nitric oxide or nitric oxide donors, super oxide dismutases, super oxide dismutase mimetic, 4-amino-2,2,6,6-tetramethylpiperidine-1-oxyl (4-amino-TEMPO), estradiol, anticancer agents, dietary supplements such as various vitamins, and a combination thereof. Examples of such cytostatic substance include angiopeptin, angiotensin converting enzyme inhibitors such as captopril (e.g. Capoten® and Capozide® from Bristol-Myers Squibb Co., Stamford, Conn.), cilazapril or lisinopril (e.g. Prinivil® and Prinzide® from Merck & Co., Inc., Whitehouse Station, N.J.). An example of an antiallergic agent is permirolast potassium. Other therapeutic substances or agents that may be appropriate include alpha-interferon, and genetically engineered epithelial cells.

Suitable cytostatic or antiproliferative agents include, without limitation, angiopeptin, angiotensin converting enzyme inhibitors such as captopril, cilazapril or lisinopril, calcium channel blockers such as nifedipine; colchicine, fibroblast growth factor (FGF) antagonists; fish oil (ω-3-fatty acid); histamine antagonists; lovastatin, monoclonal antibodies such as, without limitation, those specific for Platelet-Derived Growth Factor (PDGF) receptors; nitroprusside, phosphodiesterase inhibitors, prostaglandin inhibitors, suramin, serotonin blockers, steroids, thioprotease inhibitors, triazolopyrimidine (a PDGF antagonist) and nitric oxide.

Suitable antiallergic agents include, without limitation, permirolast potassium.

Other suitable bioactive agents include, without limitation, alpha-interferon, genetically engineered epithelial cells, dexamethasone and its derivatives, rapamycin derivatives and analogs such as 40-O-(2-hydroxyethyl)rapamycin (EVEROLIMUS®), 40-O-(3-hydroxypropyl)rapamycin, 40-O-[2-(2-hydroxyethoxy)]ethyl-rapamycin, and 40-O-tetrazolylrapamycin, synthetic inorganic and organic compounds, proteins and peptides, polysaccharides and other sugars, lipids, and DNA and RNA nucleic acid sequences having therapeutic, prophylactic or diagnostic activities, nucleic acid sequences include genes, antisense molecules which bind to complementary DNA to inhibit transcription, and ribozymes. Some other examples of suitable bioactive agents include antibodies, receptor ligands, enzymes, adhesion peptides, blood clotting factors, inhibitors or clot dissolving agents such as streptokinase and tissue plasminogen activator, antigens for immunization, hormones and growth factors, oligonucleotides such as antisense oligonucleotides and ribozymes and retroviral vectors for use in gene therapy; antiviral agents; analgesics and analgesic combinations; anorexics; antihelmintics; antiarthritics, antiasthmatic agents; anticonvulsants; antidepressants; antidiuretic agents; antidiarrheals; antihistamines; antimigrain preparations; antinauseants; antiparkinsonism drugs; antipruritics; antipsychotics; antipyretics; antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary; peripheral and cerebral; central nervous system stimulants; cough and cold preparations, including decongestants; hypnotics; immunosuppressives; muscle relaxants; parasympatholytics; psychostimulants; sedatives; tranquilizers; naturally derived or genetically engineered lipoproteins; and restenotic reducing agents.

Presently preferred bioactive agents include, but are not limited to, a corticosteroid, everolimus, an everolimus derivative, zotarolimus, a zotarolimus derivative, sirolimus, a sirolimus derivative, paclitaxel, a bisphosphonate, ApoA1, a mutated ApoA1, ApoA1 milano, an ApoA1 mimetic peptide, an ABC A1 agonist, an anti-inflammatory agent, an anti-proliferative agent, an anti-angiogenic agent, a matrix metalloproteinase inhibitor and a tissue inhibitor of metalloproteinase.

As used herein, “top-coat layer” refers to a nanoporous amorphous glass layer that is disposed as the outermost layer of a medical device.

As used herein, a material that is described as a layer “disposed over” an indicated substrate, e.g., a medical device or another layer, refers to a relatively thin coating of the material applied directly to essentially the entire exposed surface of the indicated substrate, including the outer and/or inner surfaces. The term “disposed over” may, however, also refer to the application of the thin layer of material to an intervening layer that has been applied to the substrate, wherein the material is applied in such a manner that, were the intervening layer not present, the material would cover substantially the entire exposed surface of the substrate.

As used herein, “nanoporous” refers to the presence of nanometer-sized holes, i.e., nanopores less than 1 micron in diameter, present in the amorphous glass top-coat layer. Presently preferred nanopore diameters are no larger than 100 nanometers, 75 nanometers, 50 nanometers, 25 nanometers or 10 nanometers. As used herein, “diameter” refers to the length of a hypothetical straight line between two points along the circumference of a nanopore where the straight line passes through the center of the nanopore.

The present invention also provides for the use of amorphous glass in topcoat applications. Instead of using a polymeric topcoat, nanoporous amorphous glass can be used as a topcoat to regulate release rate, if formation of such a layer occurs at relatively low temperature (e.g., <100° C. for most drugs), or to improve biocompatibility. Such a topcoat layer will have minimum drug extraction.

It is to be understood that an implantable medical device of the invention can have an amorphous glass primer layer, a nanoporous amorphous glass top-coat layer or both layers depending on the desired application. It is also to be understood that an implantable medical device of the invention can have either a polymeric reservoir layer or an amorphous glass reservoir layer, regardless of the presence of an amorphous glass primer layer or a nanoporous amorphous glass top-coat layer.

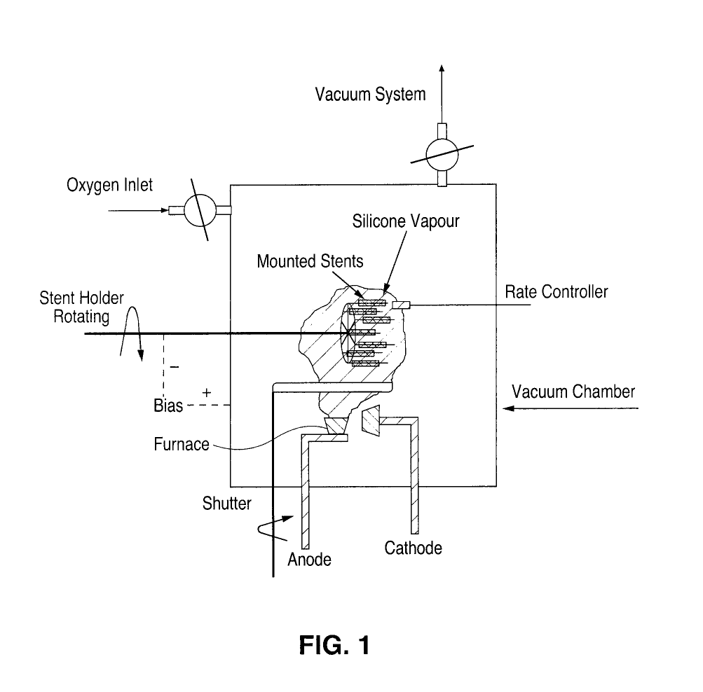

Amorphous glass is applied to either a bare metal stent or over a reservoir layer through a chemical vapor deposition process. FIG. 1 shows a schematic of an apparatus for applying amorphous glass to stents. The basic chemical reactions involved in the coating process are the following: Silicon is first sublimated above its melting temperature in order to create, under a vacuum, a significant vapor pressure. Oxygen gas is introduced into the reaction chamber using a flow controller. The oxygen reacts with silicon forming silicon oxides of different compositions, as set forth in the general chemical reaction below:

Si_{solid}→Siᵥₐₚₒᵣ+O₂(g)→SiOₓ

Evaporation of silicon is achieved by arch discharge. Since the furnace containing the silicon is placed at the anode, it is called anodic arch discharge. Ionization of the silicon vapor can be adjusted by changing the geometry of the electrode. This results in the formation of silicon plasma via the following set of chemical reactions.

Si_{solid}→Siᵥₐₚₒᵣ→Siᵥₐₚₒᵣⁿ⁺+O₂(g)→SiOₓ^{m+}→SiOₓ↓

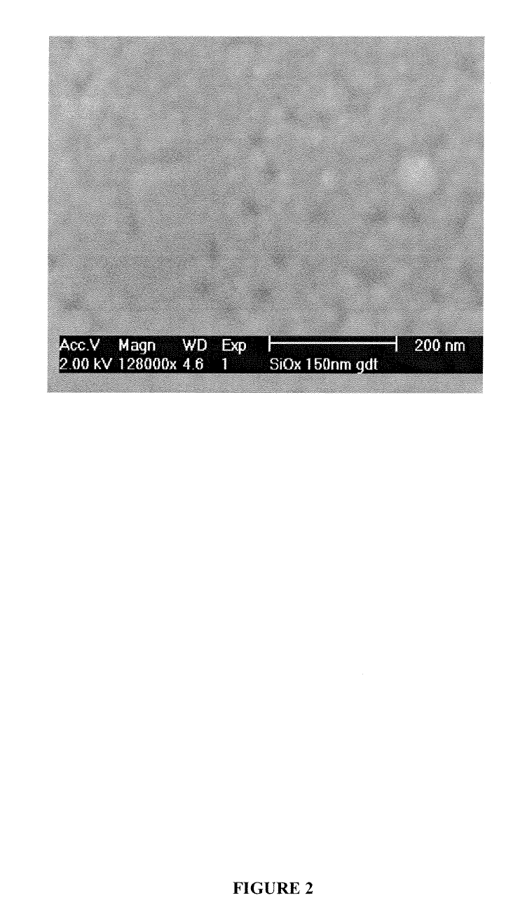

A bias field can be used to accelerate charged particles towards the stent substrate. Incoming SiOₓ^{m+} charged particles are therefore incorporated into the surface of the stent. The resulting coating is amorphous with a Vicker's hardness of 300. Amorphous glass contains nanoporosity on the scale of 25 nanometers diameter or less, as shown in FIG. 2.

The key parameters in the chemical vapor deposition process are the following: coating thickness (nm), coating composition, coating rate (nm/sec), pretreatment of the stent surface, bias potential (V), and electrode geometry. Eighteen mm-long medium Vision™ stents were coated with an amorphous glass coating. A set of fourteen experiments was conducted in order to optimize the coating process, the conditions of which are summarized in Table I. A detailed description of the coating process is described below in the Example section.

TABLE I

| | O₂ pressure | Rate | Thickness | Bias | | Composi- | |
|---|---|---|---|---|---|---|---|
| Number | [mbar] | [nm/sec] | [nm] | [V] | Pre-Treatment | tion | Stability |
| 1 | 3.0 × 10⁻³ | 4 | 220 | 350 | None | — | −− |
| 2 | 5.0 × 10⁻³ | 2 | 120 | 50 | None | — | −− |
| 3 | 5.0 × 10⁻³ | 2 | 120 | 500 | None | SiO_{0.2} | −− |
| 4 | 5.0 × 10⁻⁵ | 2 | 220 | 350 | None | — | −− |
| 5 | 5.0 × 10⁻⁵ | 2.5 | 50 | 350 | None | — | −− |
| | 3.0 × 10⁻³ | | 170 | | | | |
| 6 | 5.0 × 10⁻⁵ | 2.5 | 200 | 100 | None | SiO_{0.5} | + |
| 7 | 5.0 × 10⁻⁵ | 2.5 | 200 | 100 | None | — | −− |
| 8 | 5.0 × 10⁻⁵ | 2.5 | 50 | 100 | None | — | −+ |
| | 3.0 × 10⁻³ | | 150 | | | | |
| 9 | 5.0 × 10⁻⁵ | 2.5 | 200 | 100 | None | — | −− |
| | 3.0 × 10⁻³ | | | | | | |
| 10 | 1.5 × 10⁻³ | 4 | 100 | 100 | None | SiO_{1.4} | + |
| 11 | 5.0 × 10⁻⁵ | 2.5 | 50 | 100 | Wet cleaning | SiO_{1.3} | + |
| | 1.5 × 10⁻³ | | 50 | | | | |
| 12 | 1.5 × 10⁻³ | 2 | 100 | 100 | Wet cleaning | — | + |
| 13 | 5.0 × 10⁻⁵ | 2.5 | 50 | 350 | Wet cleaning | — | −− |
| | 1.5 × 10⁻³ | | 50 | | | | |
| 14 | 5.0 × 10⁻⁵ | 2 | 100 | 100 | Wet cleaning | SiO_{0.3} | ++ |
| | | | | | Argon discharge | | |

The present invention also provides for the use of amorphous glass as a topcoat layer since it is generally nanoporous in nature, as shown in FIG. 2. The pores are typically less than 25 nm in diameter and the pore density is such that it is porous enough to allow for drug diffusion through the layer. Thermocouple measurements have revealed that the actual stent surface temperature does not exceed 50° C. and is typically much less. This means that the coating process should not adversely affect the drug or drug/polymer layer and/or any polymer primer layers that have been applied.

The present invention further provides for amorphous glass to be used as a bioactive agent reservoir. The amorphous glass can be coated onto a medical device as described above. The device can then be immersed in a solution of bioactive agent and solvent for 1 hour, e.g., immersed in a solution that includes 1 gram of everolimus in 9 grams of methanol. The solvent can then be evaporated in a vacuum oven or a convection oven at 50° C. for 1 hour. In various embodiments, the amorphous glass reservoir layer can be coated with either a polymeric topcoat layer or a nanoporous amorphous glass topcoat layer, as described above.

The present invention can eliminate the use of currently used polymer primer layers in addition to currently used polymer reservoir and polymer topcoat layers. Elimination of any of the polymer layers can result in increased biocompatibility, improved adhesion of a primer layer to a bare metal stent, improved adhesion of a drug/polymer layer to the primer layer/stent and better control of drug release rate.

In addition, an amorphous glass coating can be used in any medical or cardiovascular device where adhesion needs to be improved, reduction of a currently used polymeric primer layer, polymeric bioactive agent layer or topcoat layer is required, drug release rate of a drug-coated medical or cardiovascular device must be controlled through use of a topcoat or a more biocompatible surface is desired.

### EXAMPLE

The following example is provided to further teach the concepts and embodiments of the present invention.

### Example 1

### Amorphous Glass-Coated Cobalt Chromium Stent

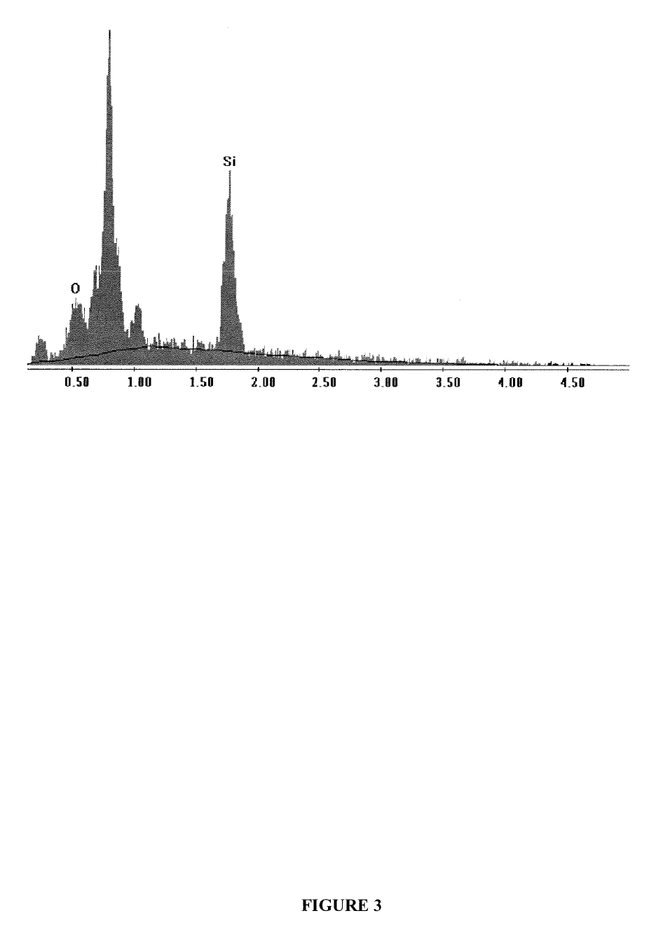

L-605 cobalt chromium stents are cleaned using a wet chemical pre-cleaning process followed by argon plasma treatment in a vacuum chamber prior to coating, both methods of which are known in the art. Cleaned stents are mounted onto a stent holder capable of holding 18 stents, as shown in FIG. 1. The holder can rotate 360° in order to allow for uniform coating of each stent with amorphous glass. The chamber is evacuated to a pressure below 1×10⁻⁵ mbar. An arch discharge is ignited while the shutter separating the stents from the silicon vapor remains in the closed position. Once the anodic arch is stable, oxygen is introduced into the chamber by a mass flow controller and monitored by the resulting pressure. After approximately 3 seconds, the reaction in the plasma is stable and the shutter is opened to the vaporous environment. Stents are then coated with the charged silicon oxide particles. Coating rate and thickness are controlled by the oxygen rate, as described above in Table I. Once the desired coating thickness is achieved, the shutter is closed, oxygen flow is stopped, and the arch discharge is turned off. After cooling, stents are removed from the chamber. Energy dispersive x-ray analysis was used on several of the coated stents confirming the chemical composition of the coating to be silicon oxide, as shown in FIG. 3.

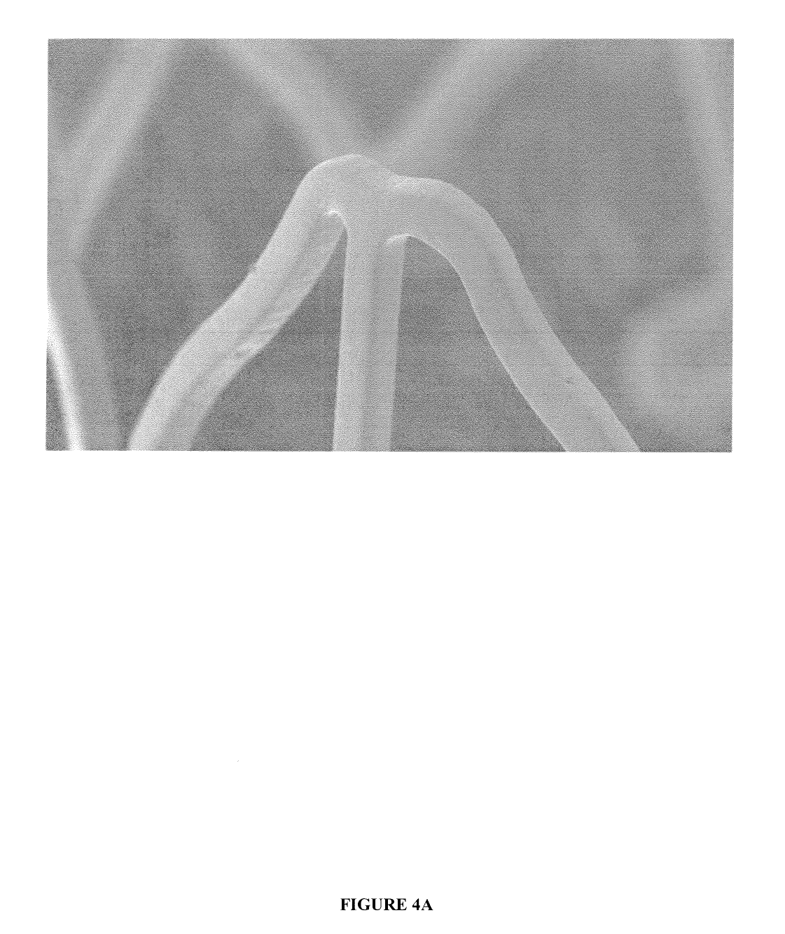

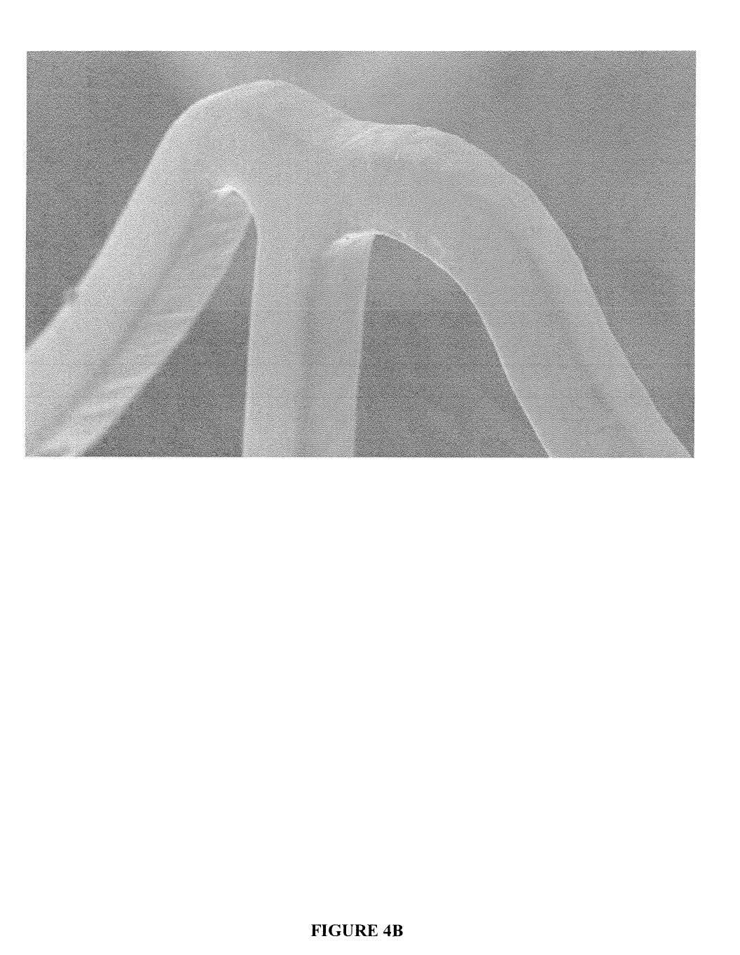

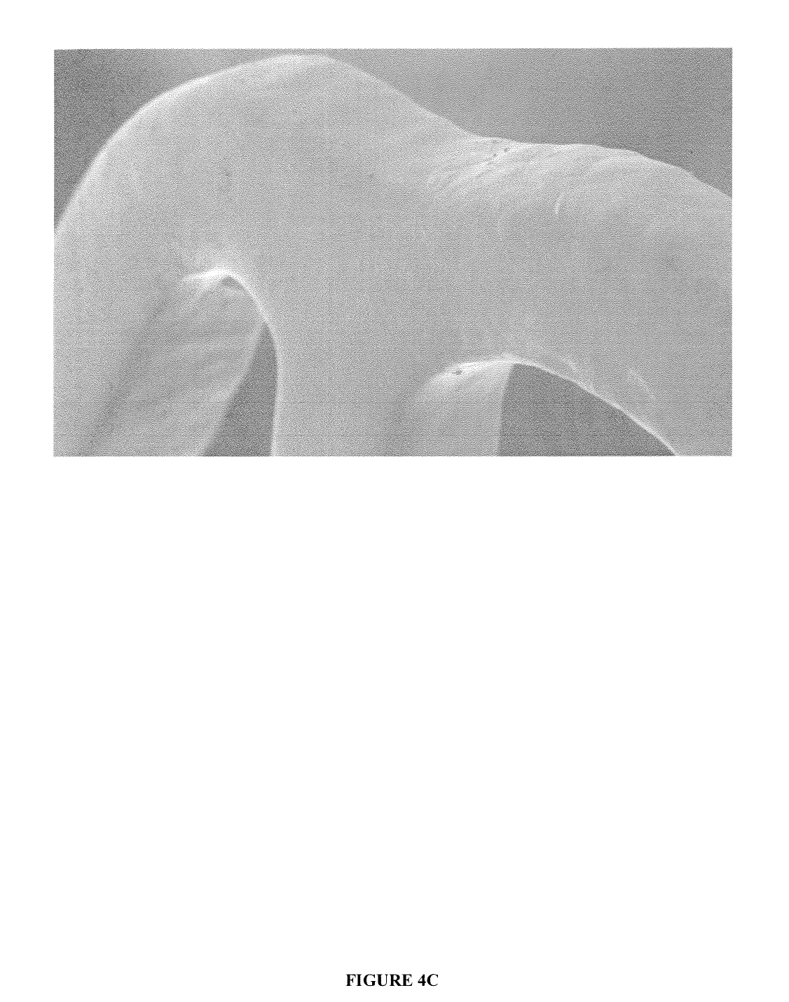

Stents produced by this process can be balloon expanded from 3-3.5 mm to 4.5 mm diameter without cracking. For example, four medium Vision™ stents, 18 mm in length were expanded, then examined for cracks using light and scanning electron microscopy. Several areas on each of the four stents were examined for cracking, delamination, flaking off and/or other issues. The areas examined included both end rings, alternating rings down the length of the stent, the “w” link, the “u” link, and the connecting link between each ring. The coating thickness of the stents was 100 nm. A typical “w” link location in one of the stents was examined at 100× magnification, shown in FIG. 4A. Examination of the region at higher magnification revealed excellent adhesion of the amorphous glass to the L-605 stent material, as shown in FIGS. 4B-C.

Using the above method, amorphous glass can also be applied to other stent materials such as stainless steel, nitinol, alloy x, tantalum and its alloys, titanium and its alloys, niobium and its alloys, and zirconium and its alloys. In addition, the coating can be applied to a metal matrix composite manufactured from any of the above pure metals and their alloys as well as to polymeric materials.

While particular embodiments of the present invention have been shown and described, it will be obvious to those skilled in the art that changes and modifications can be made without departing from this invention in its broader aspects. Therefore, the claims are to encompass within their scope all such changes and modifications as fall within the true sprit and scope of this invention.

1. An implantable medical device comprising:
a device body;
an optional amorphous glass primer layer;
a reservoir layer comprising one or more bioactive agents disposed over the device body and the amorphous glass primer layer if selected; and
an optional nanoporous amorphous glass top-coat layer disposed over the reservoir layer, wherein if the amorphous glass primer layer is not selected the nanoporous amorphous glass top-coat layer must be present and wherein if the nanoporous amorphous glass top-coat layer is not selected the amorphous glass primer layer must be present.

2. The implantable medical device according to claim 1, wherein the device body comprises a stent.

3. The implantable medical device according to claim 2, wherein the stent material is selected from the group consisting of stainless steel, nitinol, tantalum, tantalum alloy, titanium, titanium alloy, cobalt chromium, alloy x, niobium, niobium alloy, zirconium and zirconium alloy.

4. The implantable medical device according to claim 1, wherein the bioactive agent is selected from the group consisting of a corticosteroid, everolimus, an everolimus derivative, zotarolimus, a zotarolimus derivative, sirolimus, a sirolimus derivative, paclitaxel, a bisphosphonate, ApoA1, a mutated ApoA1, ApoA1 milano, an ApoA1 mimetic peptide, an ABC A1 agonist, an anti-inflammatory agent, an anti-proliferative agent, an anti-angiogenic agent, a matrix metalloproteinase inhibitor and a tissue inhibitor of metalloproteinase.

5. The implantable medical device according to claim 1, wherein the reservoir layer comprises a polymer.

6. The implantable medical device according to claim 1, wherein the reservoir layer comprises amorphous glass.

7. The implantable medical device according to claim 1, wherein the diameter of the nanopores in the nanoporous amorphous glass top-coat layer is no larger than 100 nanometers.

8. The implantable medical device according to claim 1, wherein the diameter of the nanopores in the nanoporous amorphous glass top-coat layer is no larger than 75 nanometers.

9. The implantable medical device according to claim 1, wherein the diameter of the nanopores in the nanoporous amorphous glass top-coat layer is no larger than 50 nanometers.

10. The implantable medical device according to claim 1, wherein the diameter of the nanopores in the nanoporous amorphous glass top-coat layer is no larger than 25 nanometers.

11. The implantable medical device according to claim 1, wherein the diameter of the nanopores in the nanoporous amorphous glass top-coat layer is no larger than 10 nanometers.

12. A method of coating an implantable medical device comprising:
providing an implantable medical device;
applying an amorphous glass primer layer to the implantable medical device;
applying a reservoir layer material comprising one or more bioactive agents over the amorphous glass primer layer and the implantable medical device;
applying a nanoporous amorphous glass top-coat layer over the reservoir layer material; and
forming a coating comprising the amorphous glass primer layer, the reservoir layer and the nanoporous amorphous glass top-coat layer on the implantable medical device.

13. The method according to claim 12, wherein applying the amorphous glass primer and the nanoporous amorphous glass top-coat layer comprises chemical vapor deposition.

14. The method according to claim 12, wherein the implantable medical device comprises a stent.

15. The method according to claim 14, wherein the stent material is selected from the group consisting of stainless steel, nitinol, tantalum, tantalum alloy, titanium, titanium alloy, cobalt chromium, alloy x, niobium, niobium alloy, zirconium and zirconium alloy.

16. The method according to claim 12, wherein the bioactive agent is selected from the group consisting of a corticosteroid, everolimus, an everolimus derivative, zotarolimus, a zotarolimus derivative, sirolimus, a sirolimus derivative, paclitaxel, a bisphosphonate, ApoA1, a mutated ApoA1, ApoA1 milano, an ApoA1 mimetic peptide, an ABC A1 agonist, an anti-inflammatory agent, an anti-proliferative agent, an anti-angiogenic agent, a matrix metalloproteinase inhibitor and a tissue inhibitor of metalloproteinase.

17. The method according to claim 12, wherein the reservoir layer material comprises a polymer.

18. The method according to claim 12, wherein the reservoir layer material comprises amorphous glass.

19. The method according to claim 12, wherein the diameter of the nanopores in the nanoporous amorphous glass top-coat layer is no larger than 100 nanometers.

20. An implantable medical device comprising:
a device body wherein
the device body comprises a stent having a material selected from the group consisting of stainless steel, nitinol, tantalum, tantalum alloy, titanium, titanium alloy, cobalt chromium, alloy x, niobium, niobium alloy, zirconium and zirconium alloy;
an optional amorphous glass primer layer;
a reservoir layer comprising a polymer and one or more bioactive agents disposed over the device body and the amorphous glass primer layer if selected,
wherein the bioactive agent is selected from the group consisting of a corticosteroid, everolimus, an everolimus derivative, zotarolimus, a zotarolimus derivative, sirolimus, a sirolimus derivative, paclitaxel, a bisphosphonate, ApoA1, a mutated ApoA1, ApoA1 milano, an ApoA1 mimetic peptide, an ABC A1 agonist, an anti-inflammatory agent, an anti-proliferative agent, an anti-angiogenic agent, a matrix metalloproteinase inhibitor and a tissue inhibitor of metalloproteinase; and
an optional nanoporous amorphous glass top-coat layer, having nanopores with a diameter no larger than 100 nanometers disposed over the reservoir layer,
wherein, if the amorphous glass primer layer is not selected the nanoporous amorphous glass top-coat layer must be present and if the nanoporous amorphous glass top-coat layer is not selected the amorphous glass primer layer must be present.

21. An implantable medical device comprising:
a device body wherein
the device body comprises a stent having a material selected from the group consisting of stainless steel, nitinol, tantalum, tantalum alloy, titanium, titanium alloy, cobalt chromium, alloy x, niobium, niobium alloy, zirconium and zirconium alloy;
an optional amorphous glass primer layer;
a reservoir layer comprising an amorphous glass and one or more bioactive agents disposed over the device body and the amorphous glass primer layer if selected,
wherein the bioactive agent is selected from the group consisting of a corticosteroid, everolimus, an everolimus derivative, zotarolimus, a zotarolimus derivative, sirolimus, a sirolimus derivative, paclitaxel, a bisphosphonate, ApoA1, a mutated ApoA1, ApoA1 milano, an ApoA1 mimetic peptide, an ABC A1 agonist, an anti-inflammatory agent, an anti-proliferative agent, an anti-angiogenic agent, a matrix metalloproteinase inhibitor and a tissue inhibitor of metalloproteinase; and
an optional nanoporous amorphous glass top-coat layer, having nanopores with a diameter no larger than 100 nanometers disposed over the reservoir layer,
wherein, if the amorphous glass primer layer is not selected the nanoporous amorphous glass top-coat layer must be present and if the nanoporous amorphous glass top-coat layer is not selected the amorphous glass primer layer must be present.
